(19) United States
(12) Patent Application Publication
(10) Pub. No.: US 2016/0206484 A1
(43) Pub. Date: Jul. 21, 2016

(54) WICKING YARNS FOR HYGIENE PRODUCTS

(71) Applicants: Thomas Edward SCHMITT, Concord, NC (US); Gilbert PATRICK, Kings Mountain, NC (US)

(72) Inventors: Thomas Edward SCHMITT, Concord, NC (US); Gilbert PATRICK, Kings Mountain, NC (US)

(73) Assignee: PATRICK YARNS, INC., Kings Mountain, NC (US)

(21) Appl. No.: 14/599,863

(22) Filed: Jan. 19, 2015

(51) Int. Cl. A61F 13/53

(52) U.S. Cl. CPC A61F 13/53; A61F 2013/530481; A61F 2013/530613; A61F 2013/530226; A61F 2013/530868

(57) ABSTRACT

The present invention discloses and claims an absorbent article which utilizes an adsorbent portion that includes polyamide wicking yarns, polyamide fibers, and/or polyamide fabrics with or without non-wicking yarns, fibers, or fabrics as part of the absorbent portion such that liquid is wicked to selected regions of the absorbent portion. The present invention may also include non-wicking yarns or fibers or non-woven fabrics that are primarily non-wicking cellulose. The wicking rate and fluid capacity of the polyamide wicking yarn, polyamide fibers, and/or polyamide fabrics change with the ratio of polyamide wicking and non-wicking fibers including cellulose fibers. Further, the absorbent portion may be a blend of polyamide wicking yarns, polyamide fibers and/or polyamide fabrics with non-woven wicking fabrics and/or non-wicking cellulose yarns, fibers, or fabrics.

### BACKGROUND OF THE INVENTION

1) Field of the Invention

The present invention is directed to absorbent personal care articles. In particular, the present invention is directed to polyamide wicking yarns, polyamide fibers, and polyamide fabrics incorporated into the absorbent portions of personal care articles which assist in wicking and absorbing fluids across various dimensions of the articles. Optionally the absorbent portion of the articles may also include non-wicking yarns, fibers, and fabrics. Fabrics include knitted, woven and non-woven fabrics.

2) Description of the Prior Art

Absorbent products intended to absorb discharged body fluids are well known in the art. Such absorbent products generally comprise a fibrous mass or other absorbent body which can absorb and hold the body fluids. The absorbent articles include various systems of liquid handling layers, such as intake layers, distribution layers, retention layers, and the like.

Absorbent layers often comprise superabsorbent polymer and fluffed cellulose pulp. While these materials absorb body fluids, their high absorbency is concentrated in a small region which feels wet to the user.

A yarn with wicking behavior may be incorporated into absorbent articles as disclosed in US Patent Application 2013/0158494 to Ong et al assigned to Kimberly-Clark Worldwide. In this Patent Application, the wicking yarn comprises in part superabsorbent polymer fibers. This application discloses a top sheet permeable to body fluids, for placement next to one's skin; a backsheet that is impermeable to body fluids, for placement next to an article of clothing, and an absorbent portion positioned between the top sheet and the backsheet. Superabsorbent fibers are incorporated into the absorbent portion. Although superabsorbent may be employed with the present invention, the preferred present invention does not use or employ any superabsorbent material.

Conventional absorbing systems including those with superabsorbent material have not provided the desired combination of rapid intake and distribution of liquid, surface dryness, low leakage, and comfort.

Thus a need exists for a material with rapid body fluid intake and body fluid re-distribution to create surface dryness. It is also desired that the material produce an absorbent product with low bulk and is comfortable.

### SUMMARY OF THE INVENTION

When used in an absorbent article, staple spun or continuous multifilament yarns or fabrics including non-woven fabrics comprising in part polyamide wicking and/or non-wicking fibers are used to control the degree and direction of fluid wicking Compared to typical non-woven adsorptive core materials, wetness is dispersed by capillary action and the user feels drier and more comfortable when the body fluid is dispersed.

In the broadest sense, the present invention is directed to an absorbent article having a longitudinal and transverse direction and comprising a fluid permeable topsheet, a fluid impermeable backsheet, and an absorbent portion positioned between said topsheet and said backsheet, characterized in that said absorbent portion comprises polyamide wicking yarns, polyamide wicking fibers, polyamide wicking and/or fabrics including non-woven fabrics optionally further including non-woven wicking fabric, and optionally non-wicking components such as non-wicking yarns, non-wicking fibers or non-wicking fabrics including non-woven fabrics, said polyamide wicking yarns, polyamide wicking fibers, and/or polyamide wicking fabrics including non-woven fabrics distribute fluid while non-wicking components retain and direct fluid within the absorbent portion.

### DEFINITIONS

Absorbent Article: A disposable pad of fibers and impermeable films or material worn next to the body and is designed to absorb and retain body fluids such as blood or urine.

Superabsorbent Polymer: Polymers which can absorb and retain extremely large amounts of a liquid relative to their own mass. Superabsorbent polymers are typically made from the polymerization of acrylic acid and blended with sodium hydroxide in the presence of an initiator to form polyacrylic acid sodium salt. Other materials are also used to make a superabsorbent polymer such as polyacrylamide copolymer, cross-linked carboxymethyl cellulose, or polyvinyl alcohol copolymers. Fibers may be created from some super absorbent polymers.

Yarns: For the purposes of this disclosure, the term “yarn” shall mean a length of interlocked staple fibers or continuous multifilaments suitable for use in the production of textiles or nonwoven fabrics, such as by weaving, knitting sewing, crocheting, stitching, embroidery, rope making, coating, laminating, needlepunching, water jet entangling, and adhesive, thermal or ultrasonic bonding. The term “yarn” shall include staple yarn which can be made by twisting or otherwise bonding or continuous multifilament yarn. The term yarn may for the purposes of this definition include ribbon-like materials, and strand materials having various fiber cross-sectional shapes, such as round, oval, elliptical, square, rectangular, multi-lobal, hollow, and irregularly defined shapes, as well as bicomponent fiber. The term yarn includes fiberized film yarns (i.e. slit film yarns) but does not describe a planar sheet-like material.

Fabric includes woven, non-woven, knitted, tufted, felted, braided, or bonded webs of fibers and/or yarns.

Non-Woven Fabric: Planar sheet-like fabric such as spunbond or meltblown fabric comprising fibers produced directly from melted thermoplastic polymers or polymer solutions. Non-Woven Fabric also comprises staple fibers processed on needle punched, spunlaced, or airlaid systems.

Wicking Fibers or Wicking Yarns or Wicking Fabrics: Materials with the ability to convey and disperse fluids by absorption and capillary action.

Non-Wicking Fibers, Non-Wicking Yarns, and/or Non-Wicking Fabrics: Materials which exhibit little or no fluid dispersion from the point at which fluid is applied. Thus superabsorbent fibers, yarns, or fabrics are included under this definition. Non-wicking fabrics include non-woven fabrics.

Polyamide comprises but is not limited to: Nylon 6, nylon 6-6, nylon 6-10 nylon 11, nylon 12, nylon 4-6, nylon 6-12, nylon 6-6T, nylon 6T, nylon 6I-6T, MXD6, or any combinations of these.

Polyamide Wicking Yarns, Fibers and Fabrics: Yarns, fibers and fabrics comprising polyamide wicking fiber wherein the fiber filaments wick fluids, such as body fluid(s), in the longitudinal direction of the fiber. The polyamide wicking yarns include textured, flat or staple spun polyamide wicking yarns, and optionally, a mixture or blend of polyamide wicking fibers and other wicking or non-wicking fibers. Polyamide wicking fabrics include non-woven fabrics. Polyamide wicking yarns, fibers and fabrics also include fibers with or without surface finishes applied or removed to alter the wicking characteristics of the fibers.

Non-wicking Cellulose Yarns, Fibers, and Fabrics: Fibers, yarns, and/or fabrics including non-woven fabrics comprising cleaned, natural, or bleached cotton, viscose rayon, lyocell, acetate and optionally other wicking or non-wicking fibers which are capable of forming staple spun yarns or fabrics including non-woven fabrics. Non-wicking Cellulose Yarns, Fibers, and Fabrics also include fibers with or without surface finishes applied or removed to alter the non-wicking characteristics of the fibers and/or yarns.

Cellulose Blocking Fibers or Yarns or Fabrics: Fibers, yarns, or fabrics including non-woven fabrics comprising non-wicking cellulose and/or other wicking and/or non-wicking fibers which manage and control the direction or extent of wicking, but do not wick themselves.

### BRIEF DESCRIPTION OF THE DRAWINGS

FIG. 1 is a graph of percent wet weight pickup of swine blood versus time in hours for both nylon and viscose rayon cellulose fibers.

FIG. 2 is a graph of wicking performance in terms of millimeters divided by grams of swine blood, on the vertical axis, versus time on the horizontal axis, for fibers of just nylon, a 50-50 blend of nylon and bleached cotton, a 50-50 blend of nylon and viscose rayon cellulose, solely viscose rayon cellulose, solely bleached cotton, and solely Lanseal superabsorbent fiber (available from Toyobo Corporation).

FIG. 3 is a photograph of 5 ml. swine blood applied to fibers of superabsorbent yarn showing the wicking length after a set period of time.

FIG. 4 is a photograph of 5 ml. swine blood applied to fibers of polyamide wicking yarn showing the wicking length after a set period of time.

FIG. 5 is a photograph of 5 ml. swine blood applied to cellulose yarns on the left and polyamide wicking yarns on the right showing the wicking length and blocking performance after a set period of time.

FIG. 6 is a photograph of 5 ml. swine blood applied to cellulose yarns on the left and right sides of polyamide wicking yarn (in the center) showing the wicking length and blocking performance after a set period of time.

FIG. 7 is a photograph of 5 ml. swine blood applied to yarns of 50 wt. % cellulose and 50 wt. % polyamide wicking fibers showing the wicking length after a set period of time.

### DETAILED DESCRIPTION OF THE INVENTION

The hygiene products of the present invention are designed to absorb body fluids such as urine, sweat, blood, mucus, etc. A typical hygiene product comprises a topsheet that is permeable to body fluids and comfortable for the user. The topsheet may be constructed of any woven or non-woven material which passes body fluids yet remains comfortable to the user. Suitable nonwoven materials for topsheets include, but are not limited to, hydroentangled spunlace materials, bonded carded webs made from staple fibers, and spunbond webs. Apertured films are also suitable topsheet materials. Examples of suitable topsheet materials include cellulose, bonded carded webs of polyester terephthalate, polybutylene terephthalate, polytrimethylene terephthalate, polyolefins such as polypropylene and polyethylene such as linear low-density polyethylene, low density polyethylene, high density polyethylene, etc., copolymers of polypropylene and polyethylene; polylactic acid, polyamide and/or heat-bondable fibers, including bicomponent fibers or blends or combinations thereof. Generally the topsheet is translucent to allow the user to observe the functionality of the product.

Next to the topsheet in the direction away from the body of the user is an absorbent portion comprises polyamide wicking yarns, polyamide fibers, and/or polyamide fabrics, including non-woven fabrics and optional non-wicking yarn or fibers and/or fabrics including non-woven fabrics. The polyamide wicking yarns, polyamide fibers, and/or polyamide fabrics distribute fluid within the fibrous structure length, while non-wicking components, if present, retain and direct fluid within the absorbent portion. Suitable polyamide wicking yarns, polyamide fibers, and/or polyamide fabrics are made from, but not limited to, nylon 6, nylon 6-6, nylon 6-10, nylon 11, nylon 12, nylon 4-6, nylon 6-12, nylon 6-6T, nylon 6T, nylon 6I-6T, MXD6, or any combinations of these.

Suitable non-wicking fibers include natural fibers such as cellulose such as cotton, bleached cotton, hemp, and flax, man-made cellulose fibers such as viscose rayon, lyocell, acetate; those made by animals such as silk, mink, wool, angora, and cashmere; and those man-made synthetic fibers such as polyolefin, acrylic, polyester terephthalate, polybutylene terephthalate, polytrimethyl terephthalate, polylactic acid, polyurethane, and superabsorbent polymer fiber, or a mixture of two or more of any of these non-wicking fibers. Preferred non-wicking yarns contain cellulose. All of these non-wicking fibers are capable of forming yarns or fabrics. Fabrics include woven, non-woven, knitted, tufted, felted, braided, or a bonded webs of fibers and/or yarns. Non-woven fabric includes planar sheet-like fabric such as spunbond or meltblown fabric comprising fibers produced directly from melted thermoplastic polymer or polymer solutions. Non-woven fabric also comprises staple fibers processed on needle punched, spunlaced, or airlaid systems.

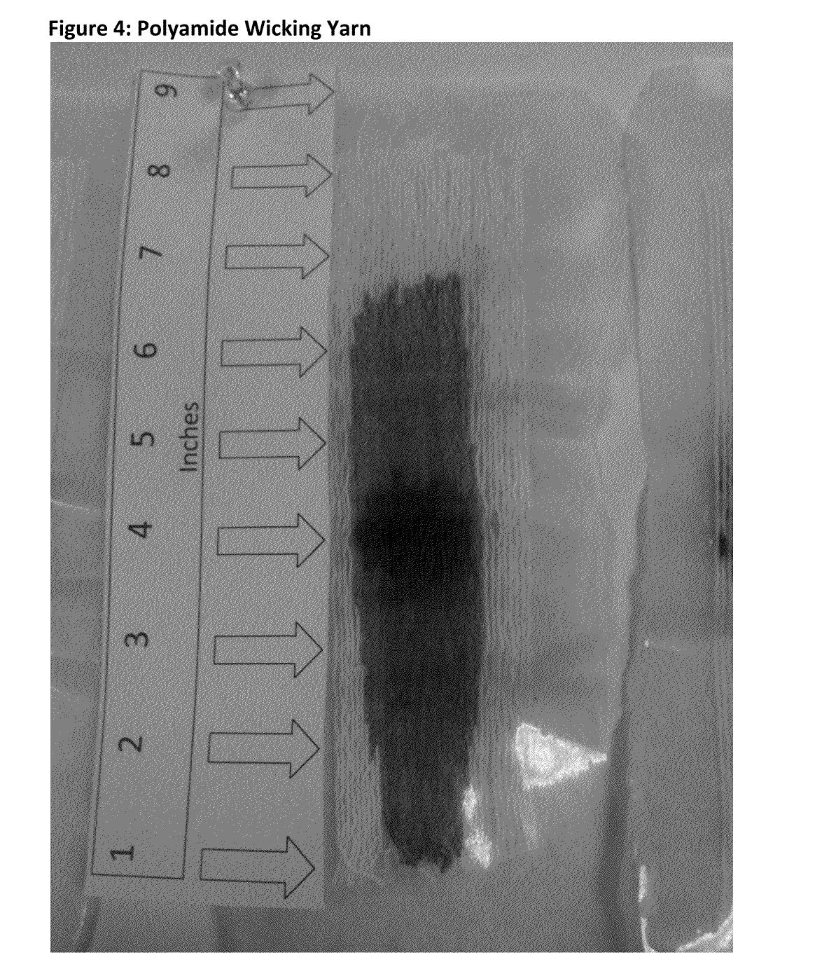

Within the scope of the present invention is a blend or combination of polyamide wicking yarns, polyamide fibers, and/or polyamide fabrics with non-wicking yarns, fibers, and/or fabrics. This combination would not wick away fluid as well as polyamide wicking yarns, polyamide fibers, and/or polyamide fabrics employed alone, but the relative amounts of wicking and non-wicking components allows those skilled in the art to create a wicking article that covers all the individual needs of the marketplace by altering the wicking length or area of the article (Note: See FIG. 4 vs. 7).

The preferred polyamide wicking fibers and the non-wicking fibers can be formed into yarns or fabrics including non-woven fabrics by using well known textile processes. There is nothing new in the process for creating the wicking yarn, fiber or fabric, nor the non-wicking yarn, fiber or fabric. Conventional staple fiber yarn processes such as ring, airjet, DREF, or open-end spinning are employed for creating the polyamide wicking and the non-wicking yarns. Conventional non-woven processes such as hydroentangled, spunlace, spunbond or bonded carded webs are employed for creating non-woven wicking and non-wicking fabrics. Although the invention will be described in terms of non-wicking cellulose fibers hereinafter, other non-wicking fibers are also included in the scope of the invention.

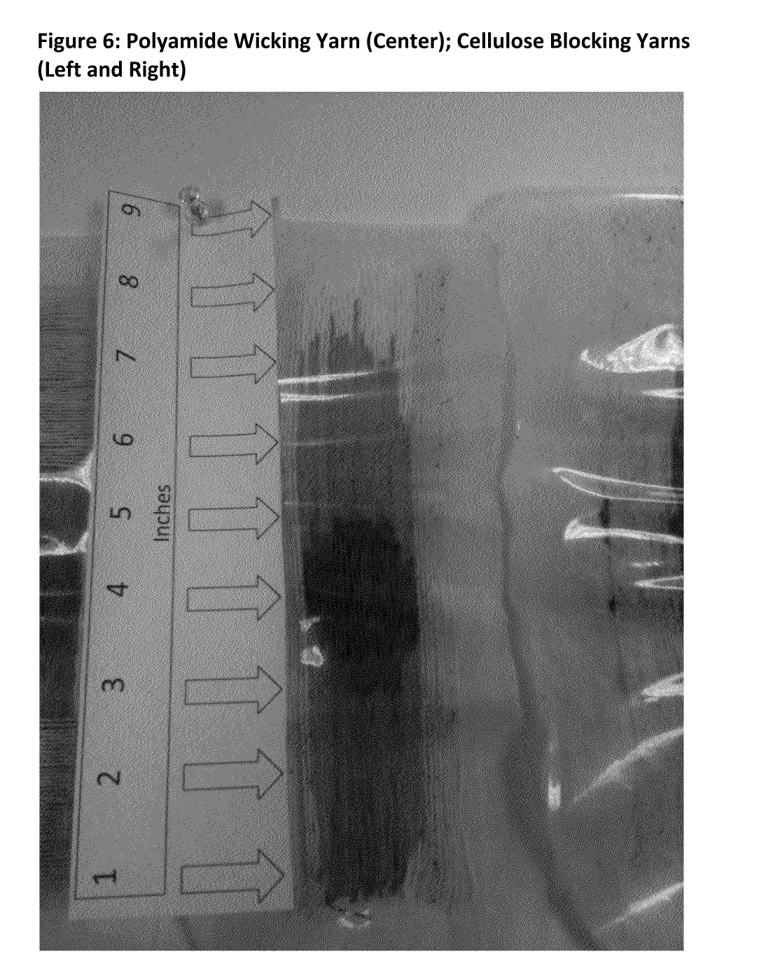

Those skilled in the art may choose yarn sizes and yarn spacing to create a range of fabric weights from about 20 to 650 grams/meter² [based upon theoretical yarn diameters for 1 cotton count (0.970 mm)** and 40 cotton count (0.154 mm) and yarns touching each other], preferably 40-500 g/m², and more preferably 60-250 g/m². For example, polyamide wicking yarns and the non-wicking cellulose yarns may weigh from about 0.59 to about 0.02 grams/meter (from 5315 to 130 denier, or from 1 to 40 cotton count) and have yarn diameters from 0.93-0.16 mm. If the yarns are positioned side-by-side according to yarn diameter on the inner surface of the back sheet (as described below) the structure will have about 5 to 70 strands of yarn per centimeter [based upon theoretical yarn diameters, above]. So if it is desired to have an absorbent component 5.0 cm. wide, there will be about 25 to 325 adjacent strands of polyamide wicking yarns and/or non-wicking yarns across the width of the absorbent component, and extending length wise. The areal weight of the fabric would be about 20 to 650 g/m². The non-wicking cellulose yarns are of similar size and weight. The non-wicking cellulose yarns are employed to prevent the flow or wicking of the body fluids in a direction not desired. Thus the non-wicking cellulose blocking yarns can surround the polyamide wicking yarns or at least be positioned on each side of the polyamide wicking yarns to retain the body fluid on the polyamide wicking yarns, such as shown in FIG. 6. **Diameter Source: Dictionary of Textile Technology; Hoechst-Celanese Corporation 1965 pg. 217.

The scope of the invention includes an alternating arrangement of polyamide wicking yarns and non-wicking yarns (such as PNPNPN) or multiple polyamide wicking yarns positioned together side-by-side with one or more non-wicking yarns (such as NNNPPPPNNPPPNNN).

While there is no minimum amount of polyamide wicking yarns, polyamide fibers or polyamide fabrics that can be employed as part of the absorbent component, the amount of the polyamide wicking fibers should be sufficient to manage the anticipated amount of fluid without transverse leakage for the length of the wicking yarn. Preferably the amount of polyamide wicking yarn is 20-100 wt. %, more preferably 50 to 100 wt. %, and most preferably 100 wt. % of the primary absorbent component. When the absorbent component comprises less than 100 wt. % of polyamide wicking yarns, polyamide fibers, or polyamide fabrics, it is preferred that the remaining be non-wicking yarns, fibers or fabrics.

Polyamide wicking yarns include staple spun yarn, continuous multifilament yarn, and slit film yarn. The continuous multifilament yarn, and slit film yarn have deniers and areal fabric weights similar to those of staple spun yarn.

Non-woven fabrics comprising polyamide wicking fibers wick but do not provide unidirectional wicking. Non-unidirectional wicking can be managed by employing non-wicking yarn and/or non-woven fabrics on either side of the polyamide non-woven fabrics to prevent transverse wicking.

Those skilled in the art may choose the composition of wicking and non-wicking yarns, fibers, and fabrics and weight, thickness, and placement to meet requirements for fluid absorption and wicking.

Also the typical product of the present invention includes a backsheet designed to be impermeable to body fluids such that body fluids are retained between the topsheet and the backsheet in the absorbent portion. The backsheet may be any suitable material known in the art, such as embossed and non-embossed films, breathable and non-breathable films, bonded nonwoven webs, laminated tissue, and combinations of the foregoing. The backsheet can be transparent, translucent or opaque depending on the product needs. For aesthetic appeal, printed features may also be included on the backsheet.

In summary the present invention can comprise:

1) an absorbent article with just polyamide wicking yarns, polyamide fibers, and/or polyamide fabrics;

2) an absorbent article with polyamide wicking yarns, polyamide fibers, and/or polyamide fabrics, with optional non-wicking yarns, fibers or fabrics positioned adjacent polyamide wicking yarns, fibers, or fabrics such as an alternating pattern, a random grouping pattern, or a center polyamide wicking yarns, fibers or fabrics positioned between regions of non-wicking yarns, fibers, or fabrics;

3) an absorbent article comprising blends of polyamide wicking yarns, polyamide fibers, and/or polyamide fabrics, with non-wicking yarns, fibers or fabrics;

4) an article having a topsheet, a backsheet and an absorbent portion there between with just polyamide wicking yarns, polyamide fibers, and/or polyamide fabrics;

5) an article having a topsheet, a backsheet, and a heterogeneous absorbent portion there between with polyamide wicking yarns, polyamide fibers, and/or polyamide fabrics, with non-wicking yarns, fibers or fabrics positioned adjacent polyamide wicking yarns, fibers, or fabrics such as an alternating pattern, a random grouping pattern, or a center polyamide wicking yarns, fibers or fabrics positioned between regions of non-wicking yarns, fibers, or fabrics; or heterogeneous layered polyamide wicking yarns, polyamide fibers, and/or polyamide fabrics with non-wicking yarns, fibers or fabrics.

6) an article having a topsheet, a backsheet and an absorbent portion there between comprising blends of polyamide wicking yarns, polyamide fibers, and/or polyamide fabrics, with non-wicking yarns, fibers or fabrics;

7) whereby any of the articles 2-3 and 5-6 incorporate non-wicking yarns, fibers, or fabrics, whereby suitable non-wicking fibers include natural fibers such as cellulose such as cotton, bleached cotton, hemp, and flax, man-made cellulose such as viscose rayon, lyocell, acetate; those made by animals such as silk, mink, wool, angora, and cashmere; and those man-made synthetic fibers such as polyolefin, acrylic, polyester terephthalate, polybutylene terephthalate, polytrimethyl terephthalate, polylactic acid, polyurethane, and superabsorbent polymer fiber, or a mixture of two or more of any of these non-wicking fibers; and

8) whereby any of the articles 1-6 include polyamide wicking yarns, polyamide fibers, and/or polyamide fabrics made from nylon 6, nylon 6-6, nylon 6-10, nylon 11, nylon 12, nylon 4-6, nylon 6-12, nylon 6-6T, nylon 6T, nylon 6I-6T, MXD6, or any combinations of these.

When incorporated into an absorbent article, the following examples, demonstrate the unexpected advantages of polyamide wicking yarns, polyamide fibers and/or polyamide fabrics with non-wicking yarns, fibers or fabrics, or blends or combinations of polyamide wicking yarns, polyamide fibers and/or polyamide fabrics with non-wicking yarns, fibers and/or fabrics to control the direction of wicking and fluid absorption in an absorbent article.

### EXAMPLE 1

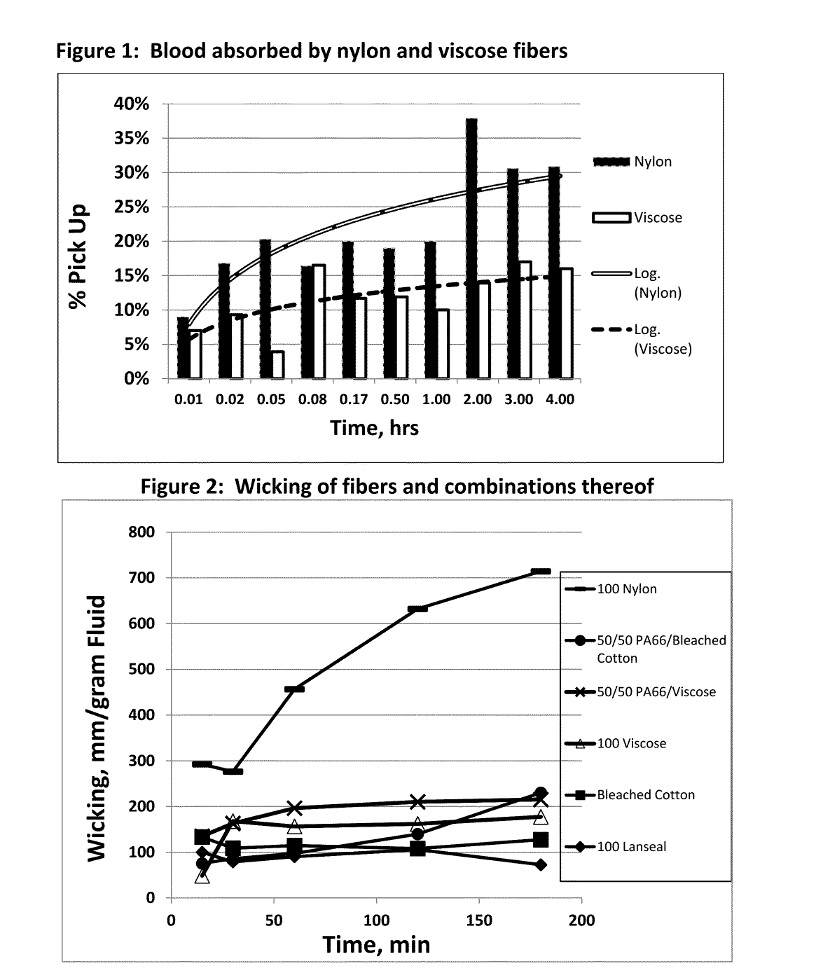

Ten fibrous strands each weighing approximately 0.25 grams/meter were prepared from polyamide 66 (Type 210 nylon available from INVISTA) and another 10 strands of similar weight of viscose rayon fibers (Modal available from Lenzing) were laid out on an impermeable horizontal surface. One milliliter of processed swine blood (available from Cocalico Biologicals) was applied at the center of each of the 10 strands of polyamide wicking and the 10 strands of viscose rayon and allowed to wick along the yarns. After fixed times as set forth in FIG. 1, the specimens were cut where the blood stopped wicking and weighed in grams. The weight of the strand was then subtracted from the weight of the blood and strand. The weight of the strand was easily calculated by measuring its length in meters and multiplying by 0.25 grams per meter. After the subtraction, this gives the weight of the blood in grams. The weight of the blood is then divided by the strand weight and multiplied by 100 to convert it to a percent. This is often referred to as “wet pickup”. FIG. 1 plots the wet pickup versus specific times. The polyamide wicking yarn strands pick up approximately twice the quantity of blood as the viscose fiber.

### EXAMPLE 2

Five fibrous strands, each weighing approximately 0.25 grams/meter were prepared from each of the following fiber compositions:
a. 100% polyamide wicking fibers
b. 50/50 polyamide/bleached cotton fibers
c. 50/50 polyamide/viscose rayon fibers
d. 100% viscose rayon fibers
e. 100% bleached cotton fibers
f. 100% Lanseal super absorbent fibers

Five strands of each specimen were laid horizontally on an impervious surface and one milliliter of swine blood was applied to the center of each strand. At fixed times as set forth in FIG. 2, a specimen of each type of strand was obtained and the blood wicking distance in millimeters was measured. Then the strand was cut at each end where the blood stopped wicking and weighed in milligrams, just as described in Example 1. This provided the total weight of the blood and strand. Again the weight of the strand was known and it was subtracted from the first weight, yielding just the weight of the blood in milligrams. This data was converted into grams by dividing by 1000. The wicking distance in mm of the next strand was then divided by its weight of the blood in grams and plotted in FIG. 2 at the 5 time periods set forth therein. FIG. 2 plots the wicking in terms of wicking length in mm divided by the grams of blood as described for each fibrous strand with time. It is a measure of wicking performance by showing how many millimeters of wicking will occur per gram of swine blood.

It is observed that the polyamide wicking strand has the greatest degree of wicking per unit weight of blood absorbed. Meanwhile, Lanseal superabsorbent fiber has the least wicking length per unit of blood absorbed. Mainly the super absorbent does not wick as it absorbs the blood. This makes the hygiene product feel wet against the skin of the user. However the broad scope of the present invention can include non-wicking fibers made from superabsorbent polymer, for example.

### EXAMPLE 3

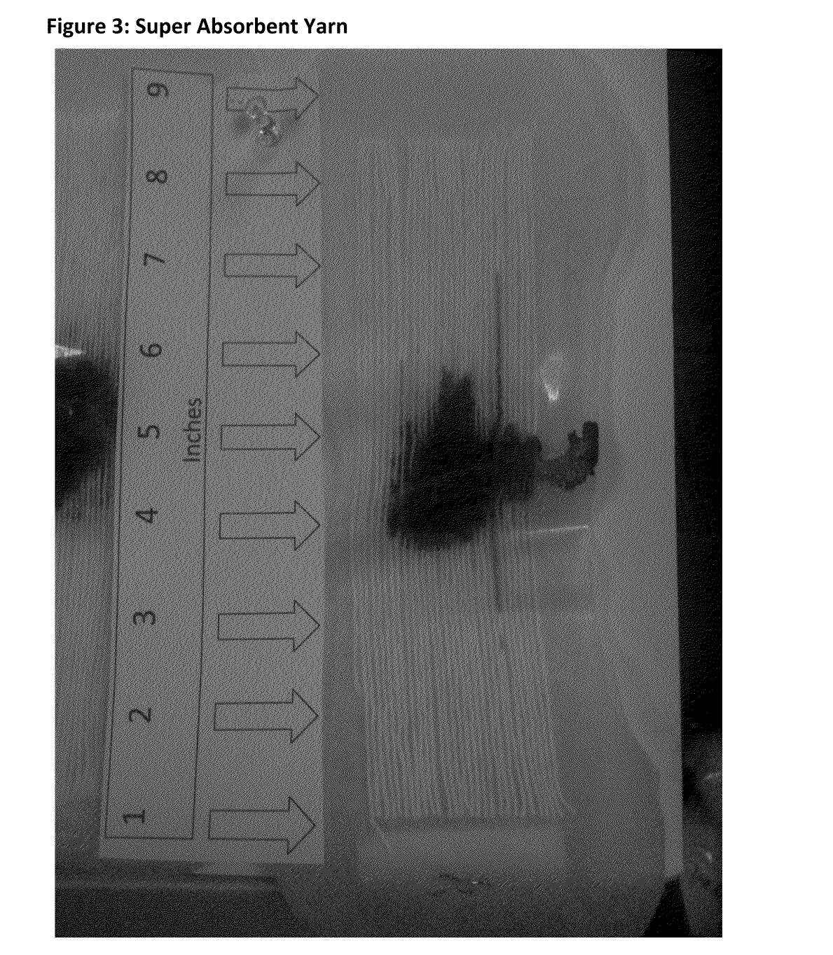

Exemplary examples of absorbent products of the claimed invention and a comparative example are found in FIGS. 3-7. The illustrative specimens for these Figures were prepared as follows:

An impervious backsheet of the absorbent product (worn against clothing) is a clear peel and stick film (available from many producers including 3M Company). The advantage of the clear film is the ability to see all fluid or dried fluid in the adsorbent article.

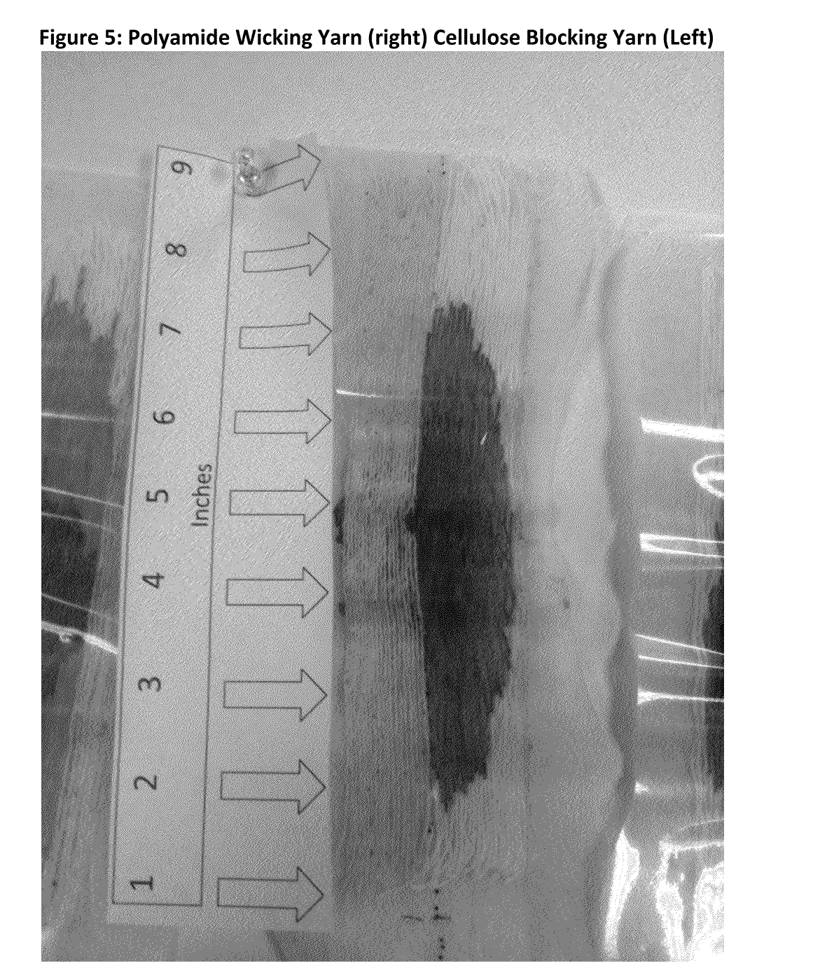

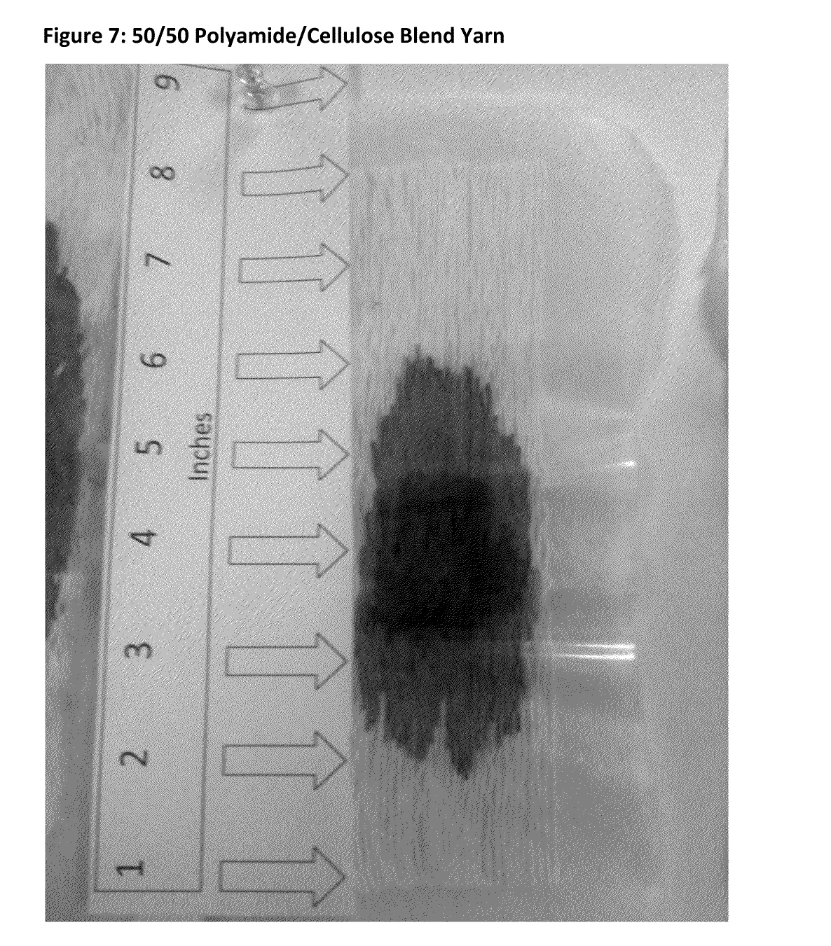

Upon the sticky side of the clear film lay 2,660 denier yarns side-by-side and touching each other. This spacing is approximately 8 yarns per centimeter. The areal weight of the yarn is approximately 250 g/m². The yarns are the absorbent member of the absorbent article. The yarns were Lanseal superabsorbent fiber in FIG. 3, polyamide wicking yarns in FIG. 4, polyamide wicking yarn on the right and non-wicking cellulose blocking yarn on the left in FIG. 5, polyamide wicking yarns in the center and non-wicking cellulose blocking yarns on both the right and left side of the polyamide wicking yarns shown in FIG. 6, and a 50-50 polyamide/cellulose blend yarn shown in FIG. 7.

On top of the absorbent core yarns is laid a polyolefin non-woven top sheet with areal weight of approximately 25 g/m². In the case of feminine care pads, the topsheet is the side worn against the body.

Test specimens are horizontally laid with the topsheet side up. To each specimen, 5 milliliters of swine blood is applied at the center of the absorbent member and the blood is allowed to wick to its maximum extent.

Inspecting the specimens, it is unexpectedly seen that 100% polyamide wicking yarns (FIG. 4) have greater wicking distance than superabsorbent yarns (FIG. 3). The polyamide wicking yarns are seen to carry away the blood and create a drier region at the challenge site.

Furthermore, it is discovered that while polyamide wicking fibers promote wicking, non-wicking cellulose fibers retard or even stop wicking FIG. 5 shows that, despite the same fluid challenge, regions of non-wicking cellulose yarns next to polyamide wicking yarns do not wick while polyamide wicking yarns do wick. The non-wicking cellulose yarns are to the left of the polyamide wicking fibers. FIG. 6 shows another example of regional flow. In FIG. 6, a region of polyamide wicking yarns is centrally positioned between two side regions of cellulose yarns. Again it is found that polyamide wicking yarns wick and cellulose yarns do not wick to any significant extent. Such a scheme may be used in an adsorbent article to prevent fluid leakage in the transverse (side-to-side) direction.

FIG. 7 shows a yarn comprising 50 percent polyamide wicking fibers and 50 percent cellulose fibers. It is seen that the degree of wicking is less than 100 percent polyamide wicking yarns shown in FIG. 4. Thus those skilled in the art upon reading this disclosure would recognize that wicking and/or non-wicking yarn composition, size and spacing or wicking and/or non-wicking non-woven fabric width, weight, and thickness can manage the direction and quantity of fluid wicked.

From the above examples, it is shown that polyamide wicking fibers are good wicking materials while cellulose fibers comparatively are not. In combination, a superior absorbent article comprising yarns of polyamide wicking and/or non-wicking fibers can be designed to move fluids to the most desirable regions.

Thus it is apparent that there has been provided, in accordance with the invention, an article that fully satisfies the objects, aims, and advantages set forth herein. While the invention has been described in conjunction with specific embodiments thereof, it is evident that many alternatives, modifications, and variations will be apparent to those skilled in the art in light of the foregoing description. Accordingly, the invention is intended to embrace all such alternatives, and modifications and variations as fall within the spirit and broad scope of the intended claims.

What is claimed is:

1. An absorbent article having a longitudinal and transverse direction and comprising: a fluid permeable topsheet, a fluid impermeable backsheet, and an absorbent portion positioned between said topsheet and said backsheet, characterized in that said absorbent portion comprises polyamide wicking yarns, and/or polyamide wicking fibers and/or polyamide wicking fabrics.

2. The absorbent article of claim 1, wherein said polyamide wicking fabrics include non-woven fabrics.

3. The absorbent article of claim 1, wherein said polyamide wicking yarns include staple spun and continuous filament yarns.

4. The absorbent article of claim 1, further including components for retaining and directing fluid including wicking or non-wicking yarns, and/or wicking or non-wicking fibers, and/or wicking or non-wicking fabrics positioned adjacent said polyamide wicking yarns, and/or polyamide wicking fibers and/or polyamide wicking fabrics.

5. The absorbent article of claim 4, wherein said non-wicking fabrics include non-woven fabrics and/or said wicking fabrics include non-woven fabrics.

6. The absorbent article of claim 1, wherein said polyamide wicking fibers, polyamide yarns, and polyamide fabrics are made from nylon 6, nylon 6-6, nylon 6-10, nylon 11, nylon 12, nylon 4-6, nylon 6-12, nylon 6-6T, nylon 6T, nylon 6I-6T, or MXD6, or any combination of these.

7. The absorbent article of claim 6, wherein said wicking fabrics include non-woven fabrics.

8. The absorbent article of claim 6, wherein the areal weight of the absorbent portion is approximately 20 to 650 g/m².

9. The absorbent article of claim 4 wherein said non-wicking yarns, non-wicking fibers and/or non-wicking fabrics contain cellulose fibers or a blend of cellulose fibers and other non-wicking fibers.

10. The absorbent article of claim 9, wherein said cellulose fibers are made from cotton, bleached cotton, viscose rayon, lyocell, or acetate, including fibers that have a non-wicking coating thereon, or a mixture of two or more of these, and said other non-wicking fibers are man-made fibers including polyolefin, acrylic, polyester terephthalate, polybutylene terephthalate, polytrimethyl terephthalate, polylactic acid, polyurethane fibers, or superabsorbent polymer fiber, and mixtures thereof, including fibers with or without surface finishes applied or removed to alter the wicking characteristics.

11. The absorbent article of claim 1, wherein said absorbent article is selected from the group comprising a feminine hygiene article including pads and tampons, an adult incontinence article, a baby diaper, a personal care article, a bandage, and a wiping towel.

12. An absorbent article having a longitudinal wicking direction, comprising: a fluid permeable topsheet, a fluid impermeable backsheet, and an absorbent portion positioned between said topsheet and said backsheet, wherein said absorbent portion comprises a combination of one or more of (a) polyamide wicking yarns or polyamide wicking fibers or polyamide wicking fabrics, and one or more of (b) non-wicking yarns or non-wicking fibers or non-wicking fabrics.

13. The absorbent article of claim 12, wherein said polyamide wicking fabrics include non-woven fabrics and/or said non-wicking fabrics include non-woven fabrics.

14. The absorbent article of claim 12, wherein said polyamide wicking yarns include staple spun and continuous filament yarns.

15. The absorbent article of claim 12, further including components for retaining and directing fluid include secondary wicking or non-wicking yarns, or secondary wicking or non-wicking fibers, or secondary wicking or non-wicking fabrics positioned adjacent said combination.

16. The absorbent article of claim 12, wherein said polyamide wicking yarns, polyamide wicking fibers, and/or polyamide wicking fabrics are made from nylon 6, nylon 6-6, nylon 6-10, nylon 11, nylon 12, nylon 4-6, nylon 6-12, nylon 6-6T, nylon 6T, nylon 6I-6T, or MXD6, or any combinations of these.

17. The absorbent article of claim 12, wherein the areal weight of the absorbent portion is approximately 20 to 650 g/m².

18. The absorbent article of claim 15, wherein said non-wicking yarns, non-wicking fibers, or non-wicking fabrics and/or secondary non-wicking yarns, secondary non-wicking fibers and secondary non-wicking fabrics contain cellulose fibers made from cotton, bleached cotton, viscose rayon, lyocell, or acetate, including fibers that have a non-wicking coating thereon, or a mixture of two or more of these, or contain man-made fibers including polyolefin, acrylic, polyester terephthalate, polybutylene terephthalate, polytrimethyl terephthalate, polylactic acid, polyurethane fibers, or superabsorbent polymer fibers and mixtures thereof, including those man-made fibers having a wicking or non-wicking treatment thereon.

19. The absorbent article of claim 12, wherein said absorbent article is selected from the group comprising of a feminine hygiene article including pads and tampons, an adult incontinence article, a baby diaper, a personal care article, a bandage, and a wiping towel.

20. The absorbent article of claim 15, wherein said secondary wicking or non-wicking yarns, fibers, or fabrics are positioned beside at least one side of said blend.
